(19) United States
(12) Plant Patent Application Publication
(10) Pub. No.: US 2003/0150030 P1
(43) Pub. Date: Aug. 7, 2003

(54) Loropetalum chinense variety rubrum named 'Melting Pot'

(76) Inventors: Barry R. Yinger, York Haven, PA (US); Thomas M. Foley JR., Katy, TX (US)

Correspondence Address: CHRISTIE, PARKER & HALE, LLP, 350 WEST COLORADO BOULEVARD, SUITE 500, PASADENA, CA, 91105, US

(21) Appl. No.: 10/061,087

(22) Filed: Feb. 1, 2002

(51) Int. Cl. A01H 5/00

(52) U.S. Cl. PLT/226

(57) ABSTRACT

A new plant variety of Loropetalum chinense var. rubrum characterized by its Burgundy to Rust colored foliage with White flowers.

### BACKGROUND OF THE INVENTION

The present invention relates to a new and distinct variety of Loropetalum chinense variety rubrum Yieh and was discovered as a branch sport of Loropetalum chinense ‘Shibori’ (unpatented).

The genus Loropetalum is included in the family Hamamelidaceae that comprises about 15 genera of shrubs and trees growing in the Northern Hemisphere. Loropetalum comprises one species of evergreen shrub or small tree, which possesses desirable ornamental characteristics. Loropetalum was first introduced to Europe as an ornamental plant the 1880s, and is believed to have been cultivated in North America since the early 1930s.

Loropetalum chinense var. rubrum is a native of China, and was first discovered in 1928 in Hunan province. It was rediscovered in the late 1980s and subsequently introduced to the United States. Loropetalum chinense var. rubrum generally has alternate, ovate leaves to 6 cm long and 3 cm wide. The plant is evergreen or partly evergreen depending on winter temperature. Leaf color ranges from light to dark Burgundy, occasionally Green, depending on light intensity, cultural conditions and cultivar. Shoots are slender, Brown, and densely pubescent, pith is solid. Leaves are generally densely pubescent overall.

Flowers of Loropetalum chinense var. rubrum are perfect with four (4) strap-like petals which are about 2 cm long and about 0.3 cm wide. Three (3) to six (6) flowers are together in each leaf axil. Pedicel and calyx have a White pubescence. Flower color is pinkish to reddish Purple depending on the cultivar. Flowering is heaviest in early spring.

### SUMMARY OF THE INVENTION

The new variety was discovered as a branch sport Loropetalum chinense ‘Shibori’ (unpatented), a White and Pink flowering plant (flowers were Pink with White striping), and differs from other cultivars known to the inventor by having Burgundy to Rust colored foliage with White flowers. Asexual reproduction of the new variety by cuttings taken in Fulshear, Tex. and Vacaville, Calif. have confirmed that the distinctive characteristics of the new variety are stable and transmitted to succeeding generations and that the new variety reproduces true to type.

### COMPARISON WITH PARENT

‘MELTING POT’ is distinguished from its parent and all other varieties of Loropetalum chinense var. rubrum of which I am aware by its Crimson to Rust colored foliage in combination with its White flowers. In contrast, the parent of ‘MELTING POT’ has Pink and White striped flowers and Green foliage.

### BRIEF DESCRIPTION OF ILLUSTRATION

The accompanying photographic illustration shows a plant of the new cultivar as true to color as is reasonably possible to make in an illustration of this character.

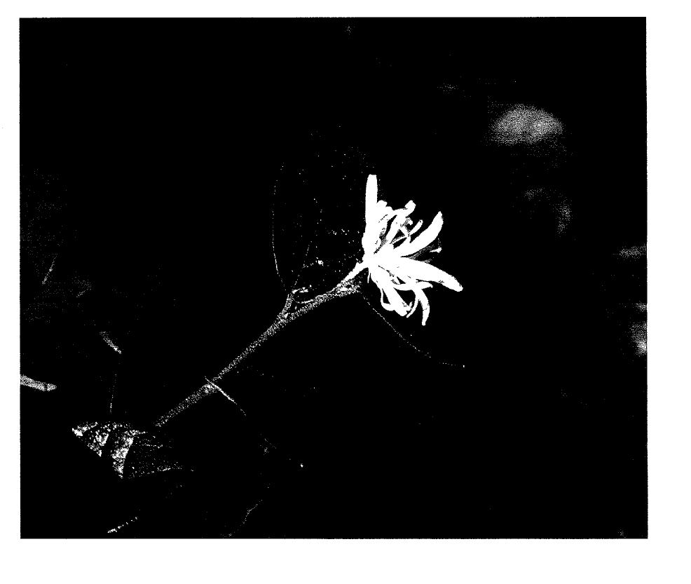

FIG. 1 illustrates the distinctive White colored flowers and Green foliage of the new variety.

### DETAILED DESCRIPTION OF THE NEW VARIETY

Loropetalum chinense var. ruburm ‘MELTING POT’ has not been observed under all possible environmental, cultural, and light conditions. The following observations and descriptions are of plants grown in Fulshear, Tex. and Vacaville, Calif. Phenotypic expression may vary with light intensity, cultural, and environmental conditions. In this description, color references are to The Royal Horticultural Society Colour Chart (2001) and terminology used in the color descriptions herein refers to plate numbers in this color chart. As with many plants in the Hamamelidaceae family, phenotypic characteristics can be dramatically different in terms of growth morphology.

Botanical Classification: Loropetalum chinense var. rubrum ‘Melting Pot’

Parentage.—Branch sport Loropetalum chinense ‘Shibori’ (unpatented).

Propagation.—By vegetative cuttings.

Plant:

Size.—Between 50 to 60 cm tall.

Habit.—Upright to obliquely ascending, to spreading for older plants.

Branching:

Branching.—Upright to obliquely ascending, spreading on older plants.

Lateral branches.—Typical Branch Quantity: About 200 Observed Branch Quantity: 10 Size: Between 50 to 60 cm long; Diameter about 6 mm.

Branch coloration.—Basal Branches: Near Greyed Group 194C Apical Branches: Near Green Group 143C Color of New Shoots: Near Green Group 143C.

Leaf:

Shape.—Lanceolate to oblanceolate.

Size.—Length about 6.5 cm; width about 2.5 cm.

Arrangement.—Alternate, simple.

Margin.—Smooth, entire.

Texture/Substance.—Coriaceus, glabrous with a waxy surface covered with punctate glands.

Color: The following color readings are of 3 year old plants grown in Vacaville, Calif. in 2 quart containers.

New foliage.—Upper side Center: Near Greyed-Green Group 191B Upper side Margin: Near White Group 155B Under side Center: Near Greyed-Green Group 191B Underside Margin: Near White Group 155B.

Mature foliage.—Upper side Center: Near Greyed-Green Group 191B Upper side Margin: Near White Group 155B Underside Center: Near Greyed-Green Group 191B Underside Margin: Near White Group 155B.

Venation pattern.—Upper Surface: Alternately pinnate, center vein depressed above Under Surface: Side veins obscure, center vein raised beneath.

Petioles.—Size: Between 3 to 4 mm Color: Near Greyed-Green Group 191B Texture: Smooth.

Internodes.—Between 2 to 3 cm.

Hardiness.—USDA Zone 6 (−10° F. to 0° F.).

Vigor.—Slow to establish; moderate. growth rate once established.

Pests/Diseases.—None noted.

Inflorescence:

Bloom period.—Outdoors: September to November Greenhouse: Not observed in a greenhouse environment.

Form.—Cyme.

Sex of flowers.—All pistilate.

Size.—Between 4 to 8 cm.

Shape.—Ovoid.

Fragrance.—None noted.

Lastingness of individual blooms.—Between 2 to 3 weeks.

Sepals.—Quantity: About 4 Shape: Broadly triangular Size: Between 1 to 2 mm.

Fruit.—Mature fruit not yet observed.

Plant:

Size.—To approximately 5 ft. tall and about 6 to 8 ft. wide.

Branching.—Dense, overall mounding to upright branching habit. Lateral Branches: Multi-branched shrub

Typical branch.—Quantity: More than 1,000 branches and stems.

Observed branch.—Quantity: More than 1,000 branches and stems Branch Size: Typical branch 1 m long, pendulous Branch Diameter: Typical branch about 1 cm in diameter; branch apex is typically 1 mm or less Branch Color: Near Grayed-Orange Group 166A Habit: Young plants exhibit pendulous growth habit. Once plant is established, plant bears obliquely upright branches.

Leaf:

Arrangement.—Simple, alternate along the stem.

Shape.—Ovate to obovate.

Apex.—Acute to obtuse.

Base.—Obtuse, occasionally unequal.

Margin.—Entire, with stellate hairs.

Size.—Young foliage: Length about 1.4 cm; width about 8 mm Mature foliage: Length 4.1 cm; width 2.7 cm.

Texture.—Adaxial: Leathery; sparsely pubescent with stellate hairs; lustrous sheen Abaxial: Leathery; sparsely pubescent with stellate hairs.

Color.—Varies with intensity of sunlight and cultural conditions; the following color readings were taken on Sep. 6, 2001 of a 2-year-old specimen in full sun of a specimen grown in Vacaville, Calif.

Young leaves.—Adaxial: Near Greyed-Purple Group 183A with margins and venation exhibiting color near Green Group 137A Abaxial: Near Greyed-Red Group 182C with midrib venation near Yellow-Green Group 147C.

Mature leaves.—Adaxial: Near Yellow-Green Group 147A; occasional mottling occurs near Yellow-Green Group 144B Abaxial: Near Green Group 138C with mid-rib near Yellow-Green Group 148C.

Venation.—Pattern: Pinnate.

Color.—Young foliage Adaxial: Near Green Group 137A Abaxial: Near Yellow-Green Group 147C Mature foliage Adaxial: Near Yellow-Green Group 144B Abaxial: Near Yellow-Green Group 148C.

Petioles.—Length: Approximately 1 to 3 mm Diameter: About 0.8 mm Color Young foliage: Near Yellow-Green Group 145B Mature foliage: Near Yellow-Green Group 146C.

Stipule length.—About 2 to 3 mm. Color: Yellow-Green Group 146C.

Internodes.—Between 0.5 to 1.5 cm long.

Hardiness.—USDA Zone 7 (0° F. to 7° F.).

Vigor.—Moderate.

Pests/Diseases.—No pest or disease problems noted.

Flower:

Habit.—Globose heads on terminals. Occasionally solitary in the leaf axils.

Bloom period.—March through April in Fulshear, Tex. with light repeat blooming throughout the growing season.

Quantity.—Between 6 to 8 flowers in a globose head.

Form.—Perfect, with 4 strap-like petals; forming a loose funnel; petal count can be as high as 6 to 8.

Diameter.—About 1.6 cm.

Height.—About 1.4 cm.

Longevity on plant.—About 4 to 5 days.

Fragrance.—None noted.

Petals.—Color Adaxial: Near White Group 155B on the margin with some coloration in the center of the petals near Red-Purple Group 69A Abaxial: Near White Group 155B Arrangement: 4 arising from calyx tube at sepal base, some flowers may have up to 8.

Shape.—Linear.

Margin.—Entire.

Apex.—Retuse.

Length.—Between 12 to 15 mm.

Width.—Between 2 to 3 mm.

Texture.—Smooth.

Bud.—Shape: Orbicular Length: About 3 mm Diameter: About 2 mm Color: Near Red-Purple Group 60B Rate of opening: About 5 to 7 days.

Peduncle.—Length: About 2 mm Diameter: About 0.5 mm Color: Near Green Group 138D Aspect: Horizontal to slightly erect in stem axils Texture: Pubescent.

Sepals.—Arrangement: Opposite; 4 per flower; Calyx erect and cruciform Shape: Ovate with a pointed apex and an entire margin Color: Near Yellow -Green Group 146C on both surfaces Length: About 2 mm Width: About 2 mm Texture: Pubescent.

Reproductive Organs:

Stamens.—4.

Pistils.—2.

Pollen.—Typical Quantity: Minute Observed Quantity: None observed Color: Off-White.

Fruit.—Type: Loculicidal capsule Quantity: Between 75 to 100 per plant per year.

Length.—About 5 mm.

Diameter.—About 6 mm.

Color.—Near Grey-Brown Group 199A.

I claim:

1. A new plant variety of Loropetalum chinense var. rubrum of the variety substantially as shown and described.
